(19) United States
(12) Patent Application Publication
(10) Pub. No.: US 2012/0150073 A1
(43) Pub. Date: Jun. 14, 2012

(54) METHOD AND APPARATUS FOR DIAGNOSING A MEDICAL CONDITION BASED UPON AUDIAL DATA FROM A PATIENT

(76) Inventors: Stephanie Dunn, Ridley Park, PA (US); George C. Newman, Philadelphia, PA (US)

(21) Appl. No.: 13/323,354

(22) Filed: Dec. 12, 2011

Related U.S. Application Data
(60) Provisional application No. 61/421,908, filed on Dec. 10, 2010.

(51) Int. Cl. A61B 7/04

(52) U.S. Cl. 600/586

(57) ABSTRACT

A system is provided that is operable to obtain acoustic data from a patient and analyze the data to diagnose whether a patient has a medical condition. In one embodiment, an acoustic detector obtains acoustic data from a patient swallowing and communicates the information with an acoustic processor. The processor analyzes the data by comparing the acoustic data to evaluate the presence of one or more acoustic features to determine the presence or absence of aspiration during swallowing.

### PRIORITY CLAIM

This application claims priority under 35 U.S.C. §119 to U.S. Patent Application No. 61/421,908 filed on Dec. 10, 2010. The entire disclosure of the foregoing application is hereby incorporated herein by reference.

### FIELD OF THE INVENTION

The present invention relates to the field of using audio data from a patient to detect and/or diagnose the presence of a medical condition. More specifically, the present invention relates to the field of recording the sound produced by a patient and evaluating the sound to detect one or more patient condition(s). In particular, the sound produced by a stroke patient while swallowing is analyzed to detect the presence of aspiration by the patient.

### BACKGROUND

There is an ongoing desire to provide medical diagnostic tools that are non-invasive, portable and relatively inexpensive. In the field of acute stroke patient care, there have been several studies that attempt to diagnose patient dysphagia and in particular patient aspiration, which is a condition that can lead to a variety of patient care issues. For instance, a stroke patient that cannot properly swallow may not be able to safely consume solid foods, which can lead to increased medical care, possible malnutrition, and/or other complications. Although studies have attempted to diagnose aspiration in stroke patients using various techniques, there exists a need for a practical, non-invasive diagnostic tool for detecting patient aspiration.

### SUMMARY OF THE INVENTION

The present invention provides a method and apparatus for detecting a medical condition based on the analysis of audio data recorded for the patient. In particular, a device detects sound produced by a patient and converts the sound into an acoustic signal. The signal is analyzed to detect one or more characteristic(s) of a medical condition.

According to one aspect, a device is provided for diagnosing the presence of a patient aspirating during swallowing. The device includes an acoustic detector for detecting the sound produced by a patient swallowing. The acoustic detector includes a transducer operable to convert the sound produced by the patient swallowing into an electronic signal. An acoustic processor processes the electronic signal from the transducer and compares features of the electronic signal to one or more pre-determined characteristic to determine whether the electronic signal correlates to a patient aspirating during swallowing.

According to another aspect, a device is provided for diagnosing the presence of a medical condition for a patient. The device includes an acoustic detector for detecting the sound produced by a patient. The acoustic detector includes a transducer operable to convert the sound produced by the patient into an electronic signal. The device also includes an acoustic processor for processing the electronic signal from the transducer to identify the portion of the electronic signal corresponding to a patient sound to be analyzed. The acoustic processor also compares data of the identified portion to one or more pre-determined acoustic characteristic to determine whether the electronic signal correlates to the presence or absence of a medical condition.

According to yet another aspect, a method is provided for diagnosing the presence of a medical condition for a patient. The method includes the step of positioning an acoustic detector onto a patient to obtain acoustic data of the sound produced by the patient when the patient swallows. The acoustic data is processed to identify the portion of the data corresponding to a portion of time relative to the patient swallowing. The identified portion of the data is analyzed to detect the presence of one or more acoustic features indicative of aspiration, and the presence of aspiration is determined in response to the step of comparing the identified portion of the data.

### DESCRIPTION OF THE DRAWINGS

The foregoing summary and the following detailed description of the preferred embodiments of the present invention will be best understood when read in conjunction with the appended drawings, in which:

FIG. 1 is cross-sectional view of an system for diagnosing a medical condition based upon audial data from a patient, incorporating an acoustic detector;

FIG. 2 is a cross-sectional view of a housing of the acoustic detector illustrated in FIG. 1;

FIG. 3 is a cross-sectional view of a retainer of the acoustic detector illustrated in FIG. 1;

FIG. 4 is a plan view of the acoustic detector illustrated in FIG. 1;

FIG. 5 is an enlarged fragmentary sectional view of the acoustic detector illustrated in FIG. 1;

FIG. 6 is a perspective view of an alternate embodiment of the acoustic detector illustrated in FIG. 1;

FIG. 7 is a graphical representation of an analysis of acoustic data obtained using the system of FIG. 6;

FIG. 8 is a graphical representation of an analysis of acoustic data obtained using the system of FIG. 6.

### DETAILED DESCRIPTION OF THE INVENTION

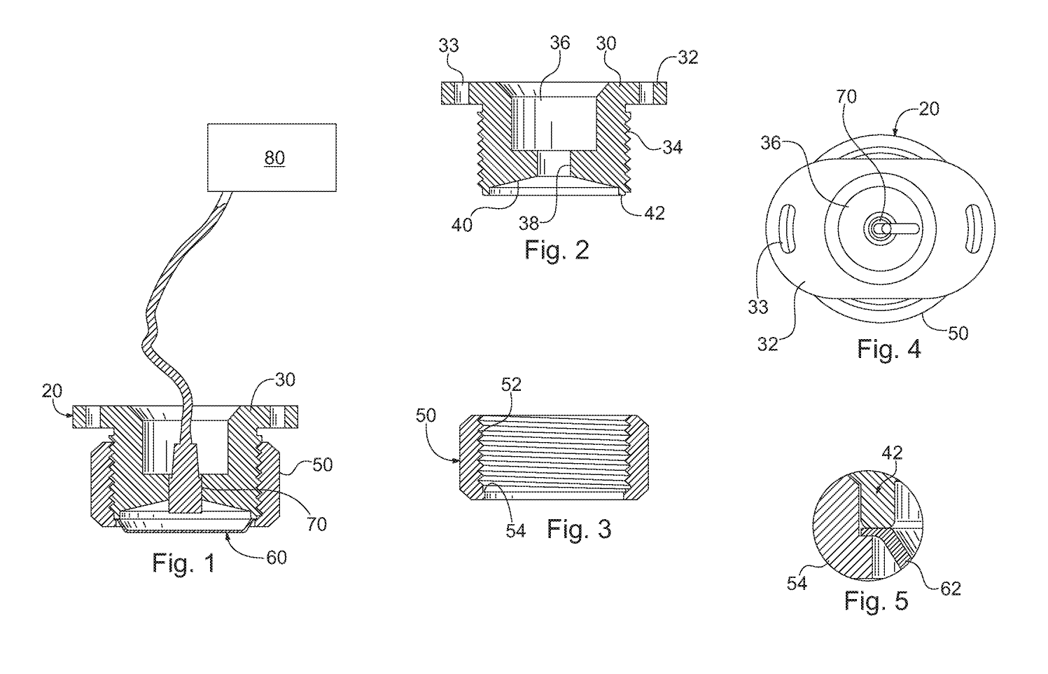

Referring now to the figures in general and to FIG. 1 specifically, a system for detecting a medical condition based on acoustic analysis is label 10. The system includes an acoustic detector 20 for detecting sound waves produced by a patient. The acoustic detector 20 includes a transducer 70 that converts the sound waves into electrical signals. An acoustic processor 80 analyzes the electrical signals to determine whether the acoustic signal indicates the presence of a medical condition.

Referring to FIG. 1-4 the details of the acoustic detector 20 will be described in greater detail. The detector 20 includes a diaphragm 60 mounted on a housing, and a retainer 50 that retains the diaphragm on the housing. The acoustic transducer 70 is mounted within the housing.

The housing 30 includes one or more elements for positioning the detector 20 on a patient. For instance, in the present instance, an enlarged flange 32 flares outwardly from the base of the housing 30. The flange 32 forms ears or tabs that project away from the housing. One or more slots or apertures are formed in the flange so that one or more straps or bands can be connected to the housing. For instance, referring to FIG. 4, the flange includes a pair of elongated slots 33 on opposing sides of the housing. A flat strap, such as a VELCRO strap having an adjustable length can pass through each of the slots and then pass around a portion of the patient, such as the throat, to hold the acoustic detector up against the patient.

The housing 30 includes an internal cavity 36 forming an opening for inserting the transducer 70 into the housing. An aperture 38 in the base of the cavity 36 forms a socket for receiving the transducer 70. A sound cavity 40 is formed at the forward end of the housing. The sound cavity comprises recessed walls that taper inwardly forming a frustoconical cavity having a major diameter forming an opening at the tip of the housing and a minor diameter at an intersection with the aperture 38. In the present instance, the walls taper at an inclusive angle of approximately 150 degrees. Adjacent the major diameter of the sound cavity, the housing forms an annular shoulder or rim 42.

The housing may include structure for connecting the diaphragm 60 to the housing. In the present instance, the housing includes an externally threaded portion 34 that cooperates with the retainer 50 to mount the diaphragm 60 on the housing.

Referring to FIG. 3, the retainer 50 is a generally cylindrical collar or ring. The retainer 50 has a connector cooperable with the housing 30 to retain the diaphragm. In the present instance, the retainer includes an internally threaded portion having threads that mate with the external threads on the housing 30. The forward end of the retainer 50 has an annular flange 54 forming a reduced diameter opening.

The retainer and the housing may be formed from a variety of materials, such as metal or plastic. In the present instance both the retainer 50 and the housing 30 are formed of a plastic, such a thermoplastic fluoropolymer. One exemplary material is a polyvinylidene fluoride plastic, such as the plastic sold under the trade name KYNAR.

The diaphragm 60 is configured to vibrate from the sounds produced by a patient. In the present instance, the diaphragm is formed from a plastic such as polytetrafluoroethylene sold by E.I. du Pont de Nemours and Company under the trade name TEFLON. However, the diaphragm may be formed of a number of other plastic or metallic materials that readily transfer the vibrations produced by sound waves from a patient. In the present instance, the diaphragm is a generally flat thin disk. A reduced thickness rim 62 extends around the circumference of the diaphragm.

Referring now to FIGS. 1 and 5, the diaphragm 60 is positioned over the sound cavity 40 of the housing 30. The rim 62 of the diaphragm 60 overlies the rim 42 on the forward end of the housing 30. The collar 50 is threaded onto the external threads 34 of the housing 30 until the annular flange 54 of the collar engages the rim of the diaphragm. In this way, the rim 62 of the diaphragm 60 is compressed between the annular flange of the collar 50 and the rim 42 of the end of the housing 30.

The sound from the patient is transmitted into the sound cavity 40 via the diaphragm 60. A transducer 70 converts the sound from the patient into an electrical signal. In the present instance, the transducer 70 is a microphone. An exemplary microphone is an omni-directional microphone such as the microphone sold by Sennheiser Electronic Corporation under model number MKE-2-PC. Although a microphone is an exemplary transducer, it should be understood that the present acoustic detector is not limited to a particular transducer. For instance, a transducer using piezoelectric crystals, such as the transducer described in U.S. Pat. No. 6,619,126 can be used instead of a microphone. Alternatively, a transducer using a capacitance sensor, such as the transducer described in U.S. Pat. No. 6,498,854 can be utilized in the acoustic detector of the present system. Accordingly, it should be understood, that the transducer of the acoustic detector may be any transducer capable of accurately converting the sound waves produced by the patient into an electrical signal.

As described above, the acoustic detector 20 is operable to detect sounds created by a patient and convert the sounds into an electrical signal. The acoustic detector 20 further includes a mechanism for communicating the electrical signal to the acoustic processor 80. In the present instance, the transducer includes a cable having a TRS jack or connector for connecting the transducer with the audio input for a computer. Although the present system uses a cable connection between the acoustic detector 20 and the acoustic processor 80, a wireless interface may be used to transmit the sound data from the transducer 70 to the acoustic processor. In such an alternative, the acoustic detector may include a transmitter for transmitting the sound data and the acoustic processor may include a receiver for receiving the sound data transmitted by the acoustic detector.

The acoustic processor 80 comprises a processor operable to process the data from the acoustic detector to detect whether the data meets one or more characteristics indicative of the presence or absence of a medical condition. For instance, in the present instance, the acoustic processor is operable to process the data to detect the presence of a sound indicative of patient dysphagia and in particular patient aspiration. The acoustic processor processes the sound data by filtering out data associated with extraneous sound, such as background noise. Additionally, the acoustic processor identifies the portion of the signal associated with the patient characteristic to be evaluated. For instance, in the present instance the acoustic processor identifies the portion of the acoustic data corresponding to the sound associated with the patient swallowing. More specifically, the acoustic processor may identify the portion of the acoustic data associated with the time interval immediate succeeding the interval associated with the patient swallowing.

After filtering the acoustic data and identifying the portion of the data corresponding to a patient characteristic to be analyzed, the acoustic processor analyzes the select data to determine the presence of one or more characteristics indicative of a medical condition. For instance, the following example illustrates the use of the system 10 to process acoustic data of patients to detect the presence of aspiration:

### EXAMPLE 1

Acoustic data was recorded for a group of patients who did not aspirate during swallowing. A second group of data was recorded for a group of patients who aspirated during swallowing. The determination of whether a patient aspirated during swallowing was made on an individual basis by a clinician observing the patient. A total of forty-six audio data samples were recorded from 30 patients.

The data was gathered for each patient by placing the acoustic detector 20 on the throat of the patient and connecting the retaining strap to position the detector on the patient's throat. The patient would then swallow a quantity of water. The acoustic detector 20 detected the acoustic signal produced by the sound of the patient swallowing and converted the sound waves into an electronic signal. The signal was output to a personal computer running Macintosh OS X based software, and the acoustic signal was recorded and stored in a file on the personal computer.

The acoustic data was then processed by an acoustic processor in the form of a personal computer. The data was processed by comparing acoustic features to determine the presence of a feature distinguishing the swallowing data from an aspirating patient from the swallowing data from a non-aspirating patient. In the present instance, two features were analyzed. The first is the Mel-frequency central coefficient (MFCC). The second feature is a statistical spectrum descriptor (SSD). For the SSD, the data was divided into short overlapping segments applying a Hanning window and the magnitude Discrete Fourier Transform (DFT) was computed.

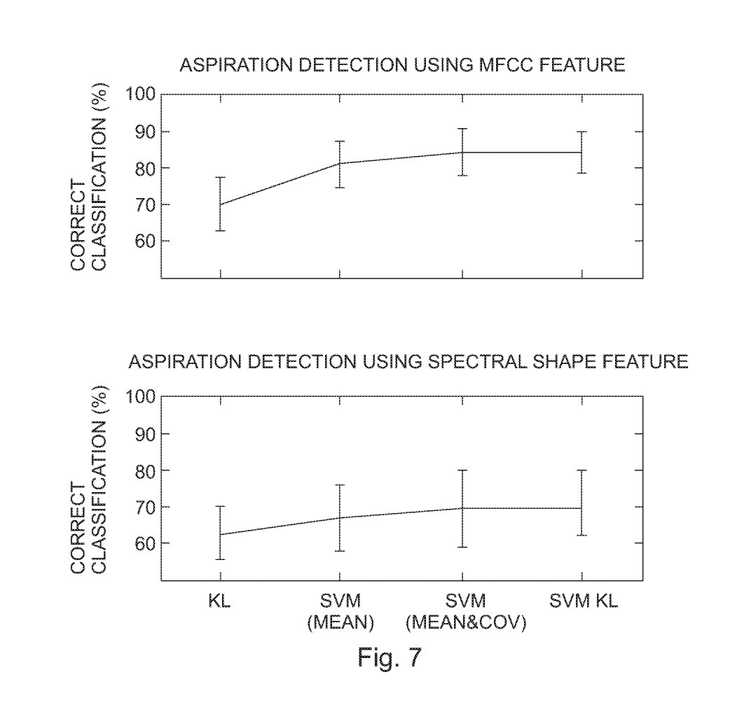

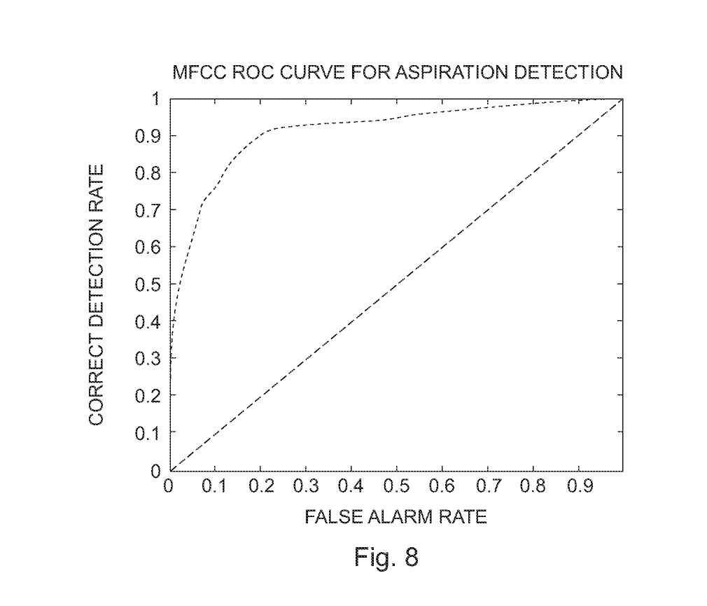

The data was then analyzed using a number of classifiers, including: (a) a distance based classification using the KL divergence; (b) support vector machines (SVMs) using only the means of the features; (c) SVM using both the means and the covariance of the features; and (d) SVMs in a KL-divergence space. The performance numbers for each classification are shown below in Table I and the results of the classification techniques are illustrated in FIGS. 7-8.

TABLE I
Aspiration Detection Performance for
Various Classification Techniques

| Feature | KL Classifier | SVM (μ) | SVM (μ, Σ) | SVM KL |
|---|---|---|---|---|
| MFCC | 69.30 ± 7.51 | 80.86 ± 6.40 | 84.21 ± 6.53 | 84.14 ± 5.78 |
| SSD | 62.30 ± 7.17 | 66.50 ± 9.32 | 69.14 ± 10.6 | 70.64 ± 8.87 |

The results show that analysis of MFCC features with SVMs provide an acceptable correlation of audio data to the presence or absence of patient aspiration. In FIG. 8 the receiver operating characteristic is illustrated, which displays the values of correct detection of each level of false alarm. From the ROC curve it will be noted that a 90% correct classification level of aspiration can be provided with a 20% level of false positives (i.e. identifying a patient as aspirating when the patient is not in fact aspirating).

Although the foregoing example provides exemplary acoustic features and classifiers, it should be understood that the analysis of the acoustic data is not limited to these features and classifiers. Additionally, in the foregoing example, the acoustic processor was the processor of a personal computer having a substantially permanent data storage element, such as a hard drive or flash memory for storing the acoustic data. It should be understood that the acoustic processor may alternatively be any of a variety of portable devices configured to receive and store an electronic signal representative of sound from a patient. For instance, the acoustic detector may interface with a portable electronic device capable of processing an audio signal, such as an mp3 file or way file. Such a portable electronic device includes an input for receiving an audio signal. For example, the input may be a built-in microphone or an input jack for connecting a microphone to the device. The device is operable to receive and store the audio signal. Additionally, the device may be able to process the acoustic signal as discussed above. Optionally, the device may store the recorded audio signal in a substantially permanent memory device, such as flash memory or a hard drive, and then communicate the audio signal with a host system, such as a personal computer as described above. The host system may include a processor for processing the audio signal as described above. The portable device may also include an element for communicating the stored file with the host system. For example, the portable device and host system may include a wireless adapter for wireless communication, such as a WiFi enabled device. Alternatively, or in addition to the wireless adapter, the host system and portable device may include a jack for connecting the portable device to the host system to transfer the audio files over the wired connection from the portable device to the remote system.

In an embodiment in which a portable device capable of processing the audio is utilized, a software program for analyzing the audio data can be loaded onto the portable device, and the acoustic detector 20 communicates the data to the electronic device. The software then processes the data to identify the subset of the data to be analyzed further for diagnosing a medical condition, such as patient swallowing as described above. Once the relevant data subset is identified, the subset is analyzed to detect the presence or absence of the medical condition. The acoustic processor then provides an indicator to the user as to whether the patient exhibits the medical characteristic or not.

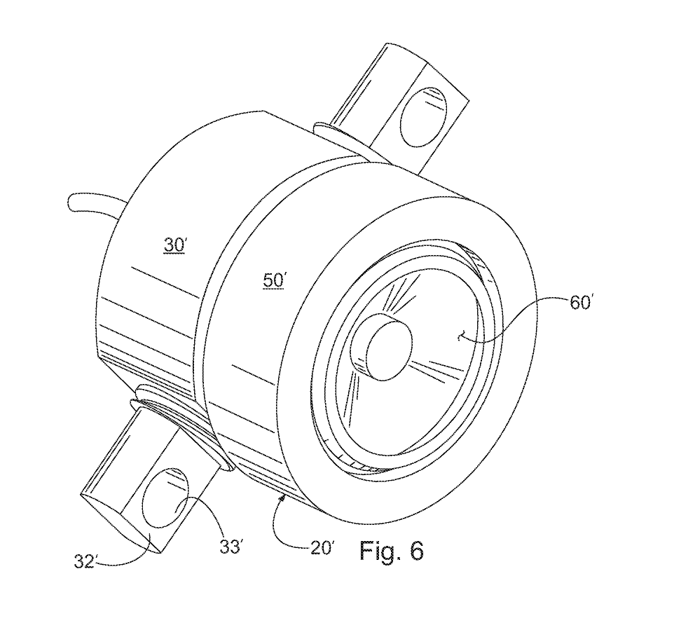

Referring to FIG. 6, an alternate embodiment of an acoustic detector 20′ is illustrated. The acoustic detector 20′ is substantially similar to the acoustic detector 20 illustrated in FIG. 1 and described above. The alternate acoustic detector has a housing 30′, retainer 50′ and diaphragm 60′, as well as a microphone for amplifying the sound wave from the patient, similar to the acoustic detector described above. The alternate acoustic detector 30′ has an alternate configuration for the element used to retain the acoustic detector 30′ on the patient during use. As can be seen in FIG. 6, the housing 30′ includes a pair of ears or tabs 32′ that project outwardly from the base of the housing 30′. The tabs 32′ are spaced apart from one another and in the present instance are on opposing sides of the housing. A hole 33′ through each tab 32′ allows a band, cord or strap to pass through each tab so that the acoustic detector can be retained on the patient during use.

Although the system has been described in connection with analyzing audial data from a patient to assess patient aspiration, it should be understood that the system may be used to diagnose a variety of medical conditions. For instance, the system may be used to detect and process audial data from a patient while sleeping and the data may be analyzed to detect a sound characteristic indicative of sleep apnea. Additionally, the system 10 may be used to detect and process audial data associated with a systolic sound over the carotid artery of a patient. The data may be analyzed to detect a sound characteristic indicative of a carotid bruit. Accordingly, it should be understood that the system and method are not limited to diagnosing a particular medical condition.

It should be noted that the acoustic processor may include an operator interface. For instance, if the acoustic processor is a personal computer, the acoustic processor may include a keyboard, mouse, and/or other input mechanism for interfacing with the acoustic processor. In this way, the operator may vary how the acoustic data is analyzed in various situations. Similarly, if the acoustic processor is part of a portable electronic device, an operator interface may be provided in the form of a touch screen, keypad or other input mechanism.

It will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention as set forth in the claims.

1. A device for diagnosing the presence of a patient aspirating during swallowing, comprising:
an acoustic detector for detecting the sound produced by a patient swallowing, comprising:
a diaphragm configured to vibrate in response to the sound produced by a patient swallowing; and
a transducer operable to convert the sound produced by the patient swallowing via the diaphragm into an electronic signal;
an acoustic processor for processing the electronic signal from the transducer and comparing features of the electronic signal to one or more pre-determined characteristic to determine whether the electronic signal correlates to a patient aspirating during swallowing.

2. The device of claim 1 wherein the acoustic processor processes the electronic signal to identify the acoustic data corresponding to the time succeeding the patient swallowing, and wherein the acoustic processor processes such acoustic data to determine whether the acoustic data correlates to a patient aspirating during swallowing.

3. The device of claim 1 comprising a retainer for attaching the acoustic detector to the patient and retaining the acoustic detector in position on the patient to obtain the electronic signal corresponding to the patient swallowing.

4. The device of claim 3 wherein the retainer comprises a band connected with the acoustic detector, wherein the band has an adjustable length for encircling the throat of the patient.

5. The device of claim 1 wherein the transducer comprises a microphone.

6. The device of claim 1 comprising a storage element for storing data of the electronic signal produced by the transducer for the sound produced by a patient, wherein the storage element is operable to separately store the data for the electronic data for the sound produced by a first patient from the data for the electronic data for the sound produced by a second patient.

7. The device of claim 1 wherein the acoustic processor comprises a filter for filtering out a portion of the electronic signal received from the transducer.

8. The device of claim 1 wherein the acoustic processor identifies a subset of data from the electronic signal, wherein the processor then compares features of the subset of data to one or more predetermined characteristics to determine whether the electronic signal correlates to a patient aspirating during swallowing.

9. A device for diagnosing the presence of a patient aspirating during swallowing, comprising:
an acoustic detector for detecting the sound produced by a patient swallowing, comprising: a transducer operable to convert the sound produced by the patient swallowing into an electronic signal;
an acoustic processor for processing the electronic signal from the transducer and comparing features of the electronic signal to one or more pre-determined characteristic to determine whether the electronic signal correlates to a patient aspirating during swallowing.

10. The device of claim 9 wherein the acoustic processor analyzes the electronic signal from the acoustic detector to identify one or more portions of the data corresponding to

11. The device of claim 9 wherein the acoustic processor processes the electronic signal to identify acoustic data corresponding to the time succeeding the patient swallowing, and wherein the acoustic processor processes such acoustic data to determine whether the acoustic data correlates to a patient aspirating during swallowing.

12. The device of claim 9 wherein the acoustic processor comprises a filter for filtering out a portion of the electronic signal received from the transducer.

13. The device of claim 9 wherein the transducer comprises a microphone.

14. The device of claim 9 wherein the acoustic processor identifies a subset of data from the electronic signal, wherein the processor then compares features of the subset of data to one or more predetermined characteristics to determine whether the electronic signal correlates to a patient aspirating during swallowing.

15. A method for diagnosing the presence of a medical condition for a patient, comprising the steps of:
positioning an acoustic detector onto a patient to obtain acoustic data of the sound produced by the patient when the patient swallows;
processing the acoustic data to identify the portion of the data corresponding to a portion of time relative to the patient swallowing;
analyzing the identified portion of the data to detect the presence of one or more acoustic features indicative of aspiration;
determining the presence of aspiration in response to the step of analyzing the identified portion of the data.

16. The method of claim 15 comprising the step of using a retainer to connect the acoustic detector to the patient.

17. The method of claim 15 wherein the step of processing the acoustic data comprises the step of identifying portions of the data meeting predetermined characteristics indicative of a patient swallowing.

18. The method of claim 15 wherein the step of analyzing the identified portion of the data comprises comparing the identified portion of the data with one or more pre-defined acoustic characteristics to determine whether the portion of the data is indicative of aspiration.
